(19) United States
(12) Patent Application Publication
(10) Pub. No.: US 2023/0210526 A1
(43) Pub. Date: Jul. 6, 2023

(54) ADMINISTRATION DEVICE INCORPORATING A DEFORMABLE STOPPER

(71) Applicant: Aktivax, Inc., Broomfield, CO (US)

(72) Inventor: Amir GENOSAR, Broomfield, CO (US)

(21) Appl. No.: 18/183,117

(22) Filed: Mar. 13, 2023

Related U.S. Application Data
(63) Continuation-in-part of application No. 17/360,850, filed on Jun. 28, 2021.
(60) Provisional application No. 63/319,141, filed on Mar. 11, 2022.
(60) Provisional application No. 63/044,896, filed on Jun. 26, 2020.

(51) Int. Cl. A61B 17/068

(52) U.S. Cl. CPC A61B 17/068; A61B 2017/00398

(57) ABSTRACT

Disclosed is an administration device able to transition between a static state and an advancing state. The administration device includes a stopper and an actuator with a post, both disposed within a hollow body that has a sidewall disposed radially within along its longitudinal length. The actuator and post can move axially within the hollow body. The stopper has a primary wall configured to exert a pressure to seal against the sidewall when the post is in a first position. This seal can provide an aseptic barrier and prevent communication of contaminating particles such as pathogens, pyrogens, viruses, bacteria, and across the seal.

### CROSS REFERENCE TO RELATED APPLICATIONS

The present application claims benefit of priority to provisional U.S. Pat. Application 63/319,141 entitled Deformable Stopper for a Syringe filed on March 11^{th}, 2022. The present application also claims benefit of priority as a continuation-in-part application to U.S. Pat. Application 17/360,850 entitled Auto-Injector With Uniform Pressure Exertion Of A Primary Container and filed on June 28^{th}, 2021, and claims benefit of priority to the parent provisional U.S. Pat. Application 63/044,896 filed on June 26^{th}, 2020. All referenced patent applications are incorporated by reference in their entirety for all they contain. In the event that the definition or use of any term or phrase differs between the present application and any incorporated by reference, the definition or use in the present application controls.

### FIELD OF THE INVENTION

The present disclosure relates to systems for storage and delivery of medical agents comprising a hollow body and a stopper disposed therewithin.

### BACKGROUND TO THE INVENTION

The art of medical syringe technology has long felt a need to reduce the breakout force required to slide the plunger of a syringe through the barrel, with many patent applications filed that attempt to address this problem. A typical rigid body container for storage of a pharmaceutical agent comprises a stopper in a cylindrical body actuated by a rod. The whole assembly may be referred to as a plunger or a piston. The term plunger may also be used to refer to the rod itself. The medical agent is drawn into the cylinder by actuation of the plunger towards the proximal end, whether to draw a fluid medication from a vial or to draw any fluid such as blood, lymph, or flowable adipose tissue from a patient. Actuation of the plunger towards the distal end then discharges the stored contents of the syringe, such as for parenteral injection, transfer into a different container, or wastage. This basic configuration dates to the 1850s, with advances to address particular challenges in certain applications and to apply advances in materials science, chemistry, and medical science being recorded with some frequency in the intervening time.

A modern problem in the art is that of piston glide, also referred to as break force, break loose force, static friction, or stiction. The stopper abuts the wall of the cylinder, and in the static state, the stopper experiences a force of static friction. When actuated into a sliding state relative to the cylinder, the stopper experiences a force of dynamic friction that is less than the force of static friction. This results in a need for the operator, such as a nurse, surgeon, pharmacist, or another medical worker, to apply a higher initial force to commence withdrawal or discharge than is required to continue said discharge. This creates risks of uneven application, unpredictable time to withdraw or discharge stored contents, or difficulty in actuating the syringe.

The problem of stiction may be especially affected by the duration of sealing engagement between the cylinder walls and the plunger, meaning it is relevant to the long-term storage of medical agents within a syringe or syringe-like container. Traditional methods known to the art to improve piston glide include surface coating and the use of silicone lubricants, but these methods may be incompatible with emerging medical therapies, including biologics such as mRNA vaccines and related technologies. Tests have shown that such methods may cause protein aggregation reducing efficacy or causing active harm. In ophthalmic injections, such lubricants have been shown to remain in the eye, which may be harmful.

### SUMMARY OF THE DISCLOSURE

The present disclosure includes a number of elements comprising an arrangement of a stopper disposed within a hollow body, such as a syringe barrel. The arrangement may include a post as part of a plunger that serves to actuate the stopper by moving axially within the hollow body towards its distal end. In a first position, the stopper may be compressed against a sidewall disposed radially within the hollow body, and in this position, the stopper may provide an aseptic barrier preventing the entry of contaminating particles such as pathogens, pyrogens, viruses, bacteria, etc. In a second position, the plunger and the post may move axially towards the stopper, contacting it centrally on a distal wall of an interior cavity such that the primary wall of the stopper may be radially contracted and axially stretched. Such actuation may reduce the compression of the primary wall against the sidewall, thereby reducing the static friction force/breakout force to translate the piston such that a storage volume distal of the stopper may be depleted. This arrangement allows for a stopper with a high initial compressive load on the sidewall, which may be used to improve the quality of the barrier provided by the stopper. The barrier may not always be configured as an aseptic barrier, but the arrangement may permit such a configuration.

As stated, a storage volume may be provided distal to the stopper within the hollow body. The actuator may comprise a post, aka a poost, extending centrally and axially relative to the sidewall, and may further extend into an interior cavity defined within the stopper. The actuator may comprise a skirt that extends radially on its distal portion and which may engage to form a seal against the cylindrical sidewall. This seal may form a sterile barrier.

### BRIEF DESCRIPTION OF THE DRAWINGS

FIG. 1a illustrates an exterior of an administration device.

FIG. 1b illustrates an initial configuration of a stopper and actuator.

FIG. 1c illustrates the previous configuration when force is applied to a stopper by an actuator.

FIG. 2a illustrates an initial configuration of an actuator that increases the initial radial compressive force of a stopper against a cylindrical sidewall.

FIG. 2b illustrates the previous configuration in which force is applied to a stopper by an actuator to cause radial deformation inward.

FIG. 3 illustrates a variation in which an actuator is made of at least two joined elements.

FIG. 4 illustrates a variation in which the stopper includes a proximal seal.

FIG. 5a illustrates a variation in which the stopper includes more than one material.

FIG. 5b illustrates a stopper.

FIG. 6 illustrates a variation in which an actuator is made of two joined elements, and a stopper includes more than one material.

FIG. 7a illustrates a variation including a pneumatic actuator.

FIG. 7b illustrates a variation including a pneumatic actuator in the advancing state.

FIG. 8 illustrates a variation, including a difference in material properties between a pinch point and a distal cavity.

FIG. 9 illustrates a variation with a post that has a cross-sectional diameter greater than a knob at its distal end.

### DETAILED DESCRIPTION OF THE INVENTION

The present disclosure relates to an administration device configured to transition between a static state and an advancing state. This includes many embodiments and variations of the core teachings, which may be recombined with one another without deviating from the scope and spirit of the invention. The administration device may include a stopper and an actuator comprising a post. The stopper and the actuator may both be disposed within a hollow body, including a sidewall disposed radially within the hollow body along its longitudinal length. The actuator and post may be configured to move axially within the hollow body. The stopper may include a primary wall configured to seal against the sidewall of the hollow body when the post is in a first position. The primary wall may apply a first radial pressure against the sidewall when the post is in the first position. In some arrangements, such sealing may provide an aseptic barrier and prevent communication of contaminating particles such as pathogens, pyrogens, viruses, bacteria, etc.

When the post is in a second position wherein it moves axially toward the stopper, it may contact a distal wall of the stopper centrally such that the primary wall radially contracts and axially stretches. Such actuation reduces the radial pressure of the primary wall against the sidewall, thereby reducing the static friction force/breakout force to move the stopper distally. In some embodiments, the post may comprise a knob configured to increase static friction when the post is in the first position.

The primary wall of the stopper may be configured to seal against the sidewall, and the seal may provide a barrier to fluid communication between a storage volume distal to the stopper within the hollow body and a volume proximal to the stopper. The volume proximal to the stopper may be disposed within the hollow body and/or may include an external volume.

The interior cavity of the stopper may distally terminate in a distal wall. The post may be configured to transition from a first position in the static state in which no axial force is applied by the post to the distal wall into a second position in the advancing state in which an axial force is applied to the distal wall by the post. The primary wall may be configured to radially contract into the interior cavity in response to axial stretching of the distal wall caused by the axial force applied to the distal wall in the advancing state. The axial force of the post may be applied distally, and the axial stretching may be in a distal direction. The interior cavity may include a pinch point disposed within the interior cavity, and the post can be configured to provide a resistance to an inward contraction of the primary wall at the pinch point in the static state. Such resistance may be removed in the advancing state, and the primary wall may optionally be arranged to contract inwards even before the post contacts the distal wall.

The post may include a knob and a poost. The knob may be configured with a first radial width, and the rod may be configured with a second radial width substantially equal to or greater than the first radial width of the knob. In such an arrangement, the rod may apply an axial force to the pinch point when the post is moved to a second position. The axial force applied to the pinch point may cause the pinch point to deflect downward, and may cause the primary wall to deflect radially inward such that the pressure of the stopper against the sidewall is reduced. The interior cavity may optionally further include a distal cavity disposed distally of the pinch point and may include a first radial cross-section. The pinch point may include a second radial cross-section less than the first radial cross-section.

The post may optionally include a knob with a first radial width and a rod with a second radial width substantially equal to or less than the first radial width. The interior cavity may further include a distal section made of a different material than that comprising the pinch point, and the distal section may be configured to accept the first radial width of the knob when the post translates in a distal direction. The knob may apply its resistance to an inward contraction in the static state at the pinch point when the post is in the first position. The knob may move into a distal cavity in the advancing state, thereby removing the resistance at the pinch point and allowing the interior cavity to contract radially inwardly around the rod. The knob may be configured to apply the axial force to the distal wall in the advancing state.

In some variations of the invention, the post may further include a skirt extending radially from the post. The skirt may be configured to seal against the sidewall and may comprise an elastomeric material. The post may be made of a more rigid material compared to the stopper. The first radial pressure applied by the stopper against the sidewall may provide an aseptic barrier between the primary wall of the stopper and the sidewall.

In some embodiments, the post may be actuated from a first position to a second position by an increase in pressure of a proximal volume acting on an actuator comprising a plunger. Such a plunger may comprise a post, knob, rod, or any other such feature as described and may be driven in a distal direction by the increase in pressure.

The actuator may comprise a secondary stopper, including the post. The actuator may be configured to apply the axial force to the distal wall through the post. In some variations, an interior surface of the primary wall includes raised surface features protruding radially inwardly into the interior cavity.

A longitudinal extent of the post may be of an axial length greater than an axial depth of the interior cavity. The post may further include a radial flange disposed proximally of the stopper. The radial flange may have a radial cross-section greater than a radial cross-section of a proximal end of the interior cavity. The arrangement of the radial flange may be such that the radial flange will axially engage the stopper in the advancing state. The post may longitudinally extend an axial distance greater than an axial depth of the interior cavity, and engagement of the radial flange with the stopper may serve to limit distal stretching of the primary wall and/or the distal wall to a predefined amount, which may be responsive to the axial force applied to the distal wall by the post in the advancing state.

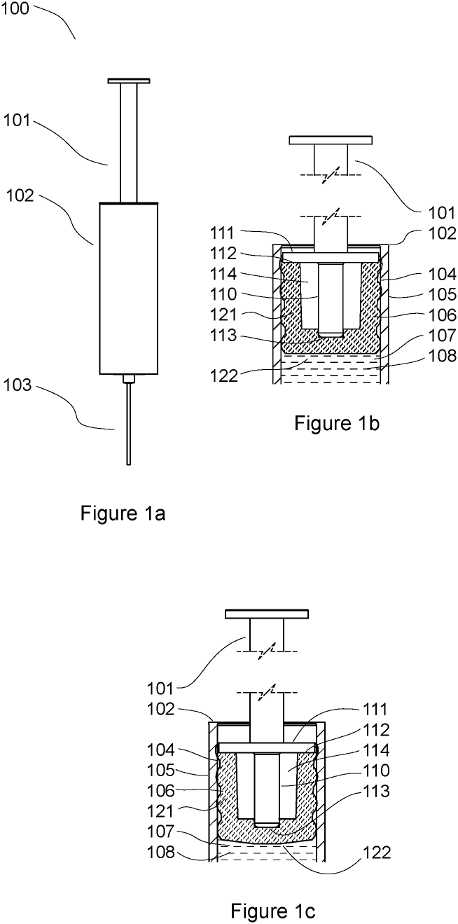

FIG. 1a illustrates an exterior view of a barrel-and-piston medical syringe such as may implement the invention. An administration device 100 such as a syringe may comprise an actuator 101 such as a plunger extending in the proximal direction, a hollow body 102 such as a cylindrical barrel through which the actuator 101 may extend along its longitudinal length, and a dispensing device 103 such as a needle for injection of a stored agent, or an intranasal, otic, topical, or oral applicator, or a cannula, or a medical device connector such as a Luer connector. FIGS. 1b & 1c each shows a cross-sectional view of the interior of an administration device 100. The depicted embodiment comprises an actuator 101, further comprising a post 110 extending axially relative to the longest dimension of the actuator 101 and hollow body 102. The post 110 may further include a flange 111 extending radially outward and disposed proximally of the stopper, the flange 111 having a radial cross-section greater than a radial cross-section of a proximal end of the interior cavity. The administration device 100 includes a flange 111, which may extend radially outward from the actuator 101. The hollow body 102 may comprise a cylindrical barrel such as a syringe barrel. As illustrated, the hollow body 102 comprises a sidewall 104 on an inner surface and an outer wall 105 on an outer surface. The sidewall 104 may be disposed radially within the hollow body 102 along all or a portion of a longitudinal length of the hollow body 102.

A stopper 106 may be disposed within the hollow body 102 distal to the actuator 101 and post 110. The stopper 106 may comprise an elastomeric material, and it may be formed to include an interior cavity 114 with a distal wall 122 at its distal end and a primary wall 121 surrounding the interior cavity 114. The primary wall 121 may exert a first pressure on the sidewall 104 when the actuator 101 and post 110 are in a first position. The administration device 100 includes a distal volume 107, which may consist of a storage volume for a beneficial agent 108, such as a fluid medication or medication component. The beneficial agent 108 is shown by shading of the distal volume 107.

In FIG. 1b, the administration device 100 is shown in an initial configuration comprising a static state in which the stopper 106 and actuator 101 may be contactingly engaged at a first contact surface 112 comprising a surface of the stopper 106 and flange 111 and a second contact surface 113 comprising a surface of the stopper 106 and the post 110. Alternatively, the actuator 101 and stopper 106 may initially be situated in an initial position in which they are not contactingly engaged, or in which they are contactingly engaged at only one of the first contact surface 112 or the second contact surface 113. FIG. 1c illustrates a configuration of the administration device 100 in an advancing state in which an axial force is applied to the actuator 101 in a distal direction through the post 110 at the second contact surface 113 to the stopper 106. The flange 111 may contact the stopper 106 at the first contact surface 112 such that the flange 111 limits the longitudinal axial distal stretching of the stopper 106. Upon application of force, the post 110 may bottom out within the interior cavity 114 defined within the stopper to cause axial stretching and radial contraction, which may flex the distal wall 122 axially downward and the primary wall 121 radially inward. This actuation results in a reduction of the first radial pressure of the primary wall 121 of the stopper 106 against the sidewall 104 to a lower second radial pressure, reducing the friction force withstanding the distal motion of the stopper 106 and actuator 101. The difference in the outward force of the stopper 106 against the sidewall 104 is shown by variation in the amount of overlap of the stopper and the sidewall.

In the advancing state, the stopper 106 and actuator 101 may glide downward in the distal direction within the hollow body 102, causing the distal volume 107 to deplete a beneficial agent 108 from the storage volume through the dispensing device 103.

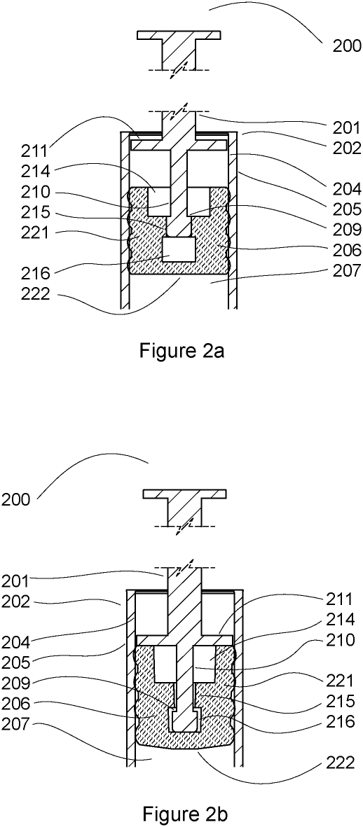

FIG. 2a illustrates an administration device 200 in an initial configuration in which an actuator 201 includes a post 210 with a knob 209 at its distal end and a flange 211 extending radially outward. The knob 209 may be positioned at a pinch point 215 in the stopper 206 such that it resists inward contraction of the primary wall 221 of the stopper 206. A hollow body 202, such as a syringe barrel, may comprise an outer wall 205 and a sidewall 204. The primary wall 221 may form a seal against the sidewall 204, and this seal may provide an aseptic barrier. In an initial position of the post 210 in the static state, the knob 209 may be configured as a protuberance or projection configured to increase the outward radial force of a stopper 206 on a sidewall 204 and increase stiction. The post 210 may further include a flange 211 extending radially outward and disposed proximally of the stopper. The flange 211 may have a radial cross-section greater than a radial cross-section of a proximal end of the interior cavity. A distal volume 207 within the hollow body 202 may form a storage volume for a fluid, including a beneficial agent such as a medication.

In some cases, the knob 209 may have the same diameter as the post 210, may still be configured to resist inward contraction of the primary wall 221 of the stopper 206, and/or may or may not include surface features that may improve resistance to inward contraction in the static state. Regarding the stopper 206, the interior cavity 214 may optionally include a distal cavity 216 disposed distally of the pinch point 215 and being of a first radial cross-section. The pinch point 215 may have a second radial cross-section less than the first radial cross-section. The pinch point 215 may be disposed within the stopper 206. The stopper 206 may include a distal wall 222 at its distal end. The pinch point 215 may be in contact with the knob 209 in the static state illustrated in FIG. 2a.

FIG. 2b illustrates the configuration of FIG. 2a in the advancing state. In this state, force is applied to an actuator 201, causing the distal end of the post 210 to move out of the pinch point 215, optionally removing resistance to radial inward contraction of the primary wall 221. The knob 209 contacts the base of the distal cavity 216 of the stopper 206 to cause radial inward contraction of the primary wall 221 of the stopper 206, thus reducing the radial pressure of the stopper 206 against the sidewall 204 from a first radial pressure which maintains a seal to a second pressure which may or may not maintain a seal. The breakout force is thus reduced in the advancing state, easing the distal motion of the stopper 206 within the hollow body 202. The difference in the outward force of the stopper 206 against the sidewall 204 is illustrated by the varying overlap of the stopper and the sidewall.

The administration device 200 may pass through an intermediate configuration between the static state of FIG. 2a and the advancing state of FIG. 2b wherein the resistance to inward contraction of the primary wall 221 is removed, but the knob 209 has not yet bottomed out against the distal wall 222.

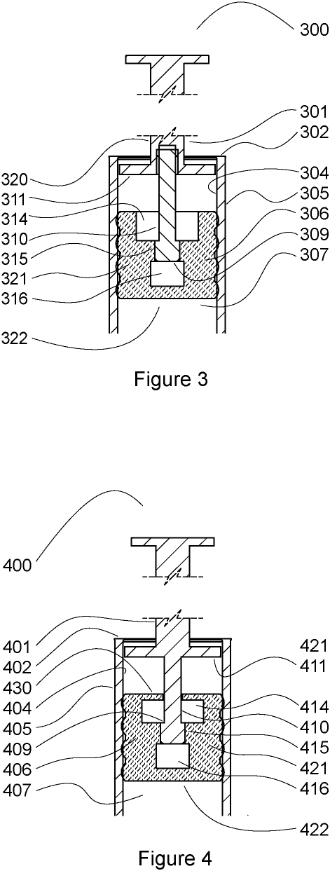

FIG. 3 illustrates an administration device 300 in which an actuator 301, such as a syringe piston assembly, may be made of two joined elements. The actuator 301 includes a post 310 extending axially relative to the longest dimension of the hollow body 302 and toward the base of a distal cavity 316 in the stopper 306 and a proximal rod 320 extending axially relative to the longest dimension of the hollow body 302 and away from the stopper 306. A distal volume 307 comprises a storage volume that may store a fluid compound or beneficial agent. Either the post 310 or the proximal rod 320 or both may comprise a flange 311 extending radially outward from the actuator 301. Such an arrangement may be advantageous for manufacturing convenience, for ease of stacking, storage, transport, or for long-term storage with a reduced risk of accidental activation or actuation by a user or handler.

The stopper 306 may comprise a pinch point 315 and a distal cavity 316. The hollow body 302 may be cylindrical and may include a sidewall 304 and an outer wall 305 extending axially along the hollow body. The post 310 may include a knob 309 (e.g., a distal knob on a distal portion or end of the post 310) configured to resist the inward contraction of a sidewall 304 of the stopper 306 and increase static friction. The knob 309 may be the same diameter as the post 310 in some variations. The post 310 may further include a flange 311 extending radially outward and disposed proximally of the stopper. The flange 311 may have a radial cross-section greater than a radial cross-section of a proximal end of the interior cavity. The stopper 306 may include an interior cavity 314 proximal to the pinch point 315. The pinch point 315 may be in contact with the knob 309 in the static state. The interior cavity 314 may include a distal cavity 316 distal to the pinch point 315.

The administration device 300 may transition between a static state, as illustrated in FIG. 3, and an advancing state, as illustrated in other embodiments of the device. The administration device 300 may pass through an intermediate configuration between the static state and the advancing state wherein the resistance to inward contraction of the primary wall 321 is removed, but the knob 309 has not yet bottomed out against the distal wall 322.

In the advancing state, the knob 309 may contact the base of the distal cavity 316 of the stopper 306 to cause radial inward contraction of the primary wall 321 of the stopper 306, thus reducing the radial pressure of the primary wall 321 of the stopper 306 against the sidewall 304 from a first radial pressure which maintains a seal to a second pressure which may not maintain a seal. The breakout force may thus be reduced in the advancing state, allowing for the distal motion of the whole plunger arrangement, including the actuator 301 and stopper 306, within the hollow body 302.

FIG. 4 illustrates an administration device 400 wherein the stopper 406 comprises a proximal wall 430. This feature may allow the actuator 401 to move the stopper 406 in a proximal direction within the hollow body 402, thus increasing the distal volume 407. The actuator 401 may comprise a syringe plunger and may include a post 410 and a knob 409 configured to resist radial inward contraction of the primary wall 421 of the stopper 406 on the sidewall 404 of the hollow body 402. This may increase stiction and may be configured to provide an aseptic barrier, which may be of benefit for the long-term storage of substances in the distal volume 407. The distal volume 407 comprises a storage volume for a medicament or other agent. The actuator 401 may comprise a flange 411 extending radially outward.

The stopper 406 may include an interior cavity 414 proximal to the pinch point 415 and a distal cavity 416 distal to the pinch point 415. The distal cavity 416 includes a distal wall 422. The hollow body 402 may be cylindrical and may include a sidewall 404 and an outer wall 405 extending axially along the hollow body. The post 410 may include a knob 409 (e.g., a distal knob on a distal portion or end of the post 410) configured to resist the inward contraction of a sidewall 404 of the stopper 406 and increase static friction. The knob 409 may be the same diameter as the post 410 in some variations. The post 410 may further include a flange 411 extending radially outward and disposed proximally of the stopper, the flange 411 having a radial cross-section greater than a radial cross-section of a proximal end of the interior cavity. The pinch point 415 may be in contact with the knob 409 in the static state. The interior cavity 414 may optionally include a distal cavity 416 disposed distally of the pinch point 415. The interior cavity 414 may have a first radial cross-section. The pinch point 415 may have a second radial cross-section less than the first radial cross-section.

The administration device 400 may transition between a static state, as illustrated in FIG. 4, and an advancing state, as illustrated in other embodiments of the device. The administration device 400 may pass through an intermediate configuration between the static state and the advancing state wherein the resistance to inward contraction of the primary wall 421 is removed, but the knob 409 has not yet bottomed out against the distal wall 422.

In the advancing state, the knob 409 may contact the base of the distal cavity 416 of the stopper 406 to cause radial inward contraction of the primary wall 421 of the stopper 406, thus reducing the radial pressure of the primary wall 421 of the stopper 406 against the sidewall 404 from a first radial pressure which maintains a seal to a second pressure which may not maintain a seal. The breakout force may thus be reduced in the advancing state, allowing for the distal motion of the whole plunger arrangement, including the actuator 401 and stopper 406, within the hollow body 402.

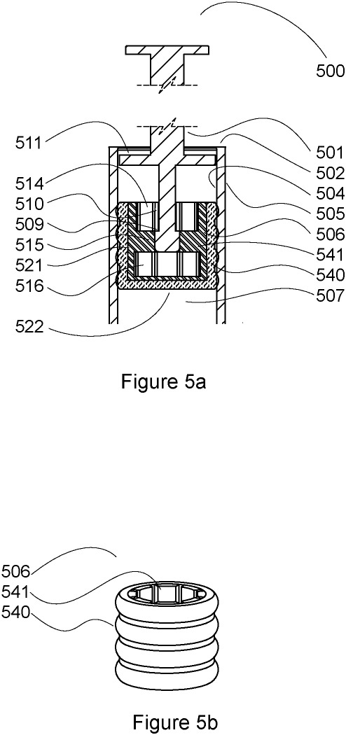

FIG. 5a illustrates an administration device 500 in which the primary wall 521 of the stopper 506 comprises an exterior surface 540 and an interior surface 541, which may be of different material compositions relative to each other. The interior surface 541 may comprise raised surface features which may be radially symmetric and configured to allow deformation of the primary wall 521 radially inward. A distal volume 507 may serve as a storage volume for a medicament or other agent. A knob 509 may be a distal protuberance situated at the distal end of the post 510 and may be configured to increase the outward force of the stopper 506 on the sidewall 504 in the static state before actuation of an actuator 501. The knob 509 may be of the same radius or larger than the post 510. A distal volume 507 comprises a storage volume of general utility. The actuator 501 may be a plunger rod comprising a flange 511 extending radially outward. The geometry of the stopper 506 may include protrusions from the primary wall 521, which may be radially symmetric around the axis of the stopper. Such protrusions may be arranged such that the primary wall 521 can deform radially inward when a force is applied to the distal wall 522 in the advancing state.

The stopper 506 may include an interior cavity 514 proximal to the pinch point 515 and a distal cavity 516 distal to the pinch point 515. The distal cavity 516 can have a distal wall 522. The hollow body 502 may be cylindrical and may include a sidewall 504 and an outer wall 505 extending axially along the hollow body. The post 510 may include a knob 509 (e.g., a distal knob on a distal portion or end of the post 510) configured to resist the inward contraction of the primary wall 521 of the stopper 506 and increase static friction of the stopper 506 against the sidewall 504. The knob 509 may be the same diameter as the post 510 in some variations. The post 510 may further include a flange 511 extending radially outward and disposed proximally of the stopper. The flange 511 may have a radial cross-section greater than a radial cross-section of a proximal end of the interior cavity. The pinch point 515 may be in contact with the knob 509 in the static state. The interior cavity 514 may optionally further include a distal cavity 516 disposed distally of the pinch point 515. The distal cavity 516 may have a first radial cross-section. The pinch point 515 may have a second radial cross-section less than the first radial cross-section.

The administration device 500 may transition between a static state, as illustrated in FIG. 5a, and an advancing state, as illustrated in other embodiments of the device. The administration device 500 may pass through an intermediate configuration between the static state and the advancing state wherein the resistance to inward contraction of the primary wall 521 is removed, but the knob 509 has not yet bottomed out against the distal wall 522.

In the advancing state, the knob 509 may contact the distal wall 522 of the distal cavity 516 of the stopper 506 to cause further radial inward contraction of the primary wall 521 of the stopper 506, thus reducing the radial pressure of the primary wall 521 of the stopper 506 against the sidewall 504 from a first radial pressure which maintains a seal to a second pressure which may or may not maintain a seal. The breakout force may thus be reduced in the advancing state, allowing for the distal motion of the whole plunger arrangement, including the actuator 501 and stopper 506, within the hollow body 502.

FIG. 5b illustrates the exterior surface 540 and the proximal opening of an interior cavity 514 defined in a stopper 506, as that illustrated by FIG. 5a. In some embodiments, the arrangement of the stopper 506 may alternatively be characterized as a single cavity comprising the interior cavity 514 and the distal cavity 516, the pinch point 515 being a perforation in a partitioning element.

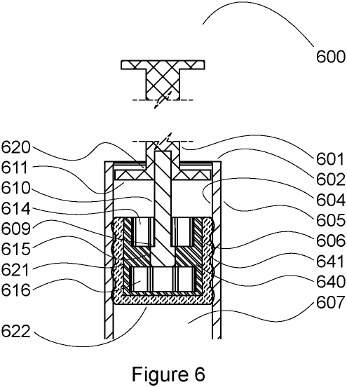

FIG. 6 illustrates an administration device 600 in which an actuator 601 comprising a syringe piston is made of two joined elements. These elements may comprise a rod 620 and a post 610, and either element may comprise a flange 611 extending radially outward. The administration device 600 may be a syringe and may include a hollow body 602, such as a cylindrical barrel. The hollow body 602 may include a sidewall 604 and an outer wall 605. A distal volume 607 may serve as a storage volume for a medicament or other agent. A stopper 606 may comprise an interior cavity 614 proximal to a pinch point 615, which may contactingly engage a knob 609. The stopper 606 may include a distal cavity 616 distal to the pinch point and interior cavity 614. The stopper 606 may have an outer surface 640 and an inner surface 641, which may have different material properties. Material properties may differ with respect to tensile strength, Young’s modulus, compressive strength, rigidity, composition, isometry, &c.

The interior cavity 614 of the stopper 606 may optionally further include a distal cavity 616 disposed distally of the pinch point 615. The distal cavity 616 may have a first radial cross-section. The pinch point 615 may have a second radial cross-section less than the first radial cross-section. The distal cavity 616 can have a distal wall 622. The hollow body 602 may be cylindrical and may include a sidewall 604 and an outer wall 605, each extending axially along the hollow body. The post 610 may include a knob 609 configured to resist the inward contraction of the primary wall 621 of the stopper 606 and maintain a high static friction of the stopper 606 against the sidewall 604. The knob 609 may be the same diameter as the post 610 in some variations. The post 610 or rod 620 may further include a flange 611 extending radially outward and disposed proximally of the stopper. The flange 611 may have a radial cross-section greater than a radial cross-section of a proximal end of the interior cavity. The pinch point 615 may be in contact with the knob 609 in the static state.

The administration device 600 may transition between a static state, as illustrated in FIG. 6, and an advancing state, as illustrated in other embodiments of the device. The administration device 600 may pass through an intermediate configuration between the static state and the advancing state wherein the resistance to inward contraction of the primary wall 621 is removed, but the knob 609 has not yet bottomed out against the distal wall 622.

In the advancing state, the knob 609 may contact the distal wall 622 of the distal cavity 616 of the stopper 606 to cause further radial inward contraction of the primary wall 621 of the stopper 606, thus reducing the radial pressure of the primary wall 621 of the stopper 606 against the sidewall 604 from a first radial pressure which maintains a seal to a second pressure which may or may not maintain a seal. The breakout force may thus be reduced in the advancing state, allowing for the distal motion of the whole plunger arrangement, including the actuator 601 and stopper 606, within the hollow body 602.

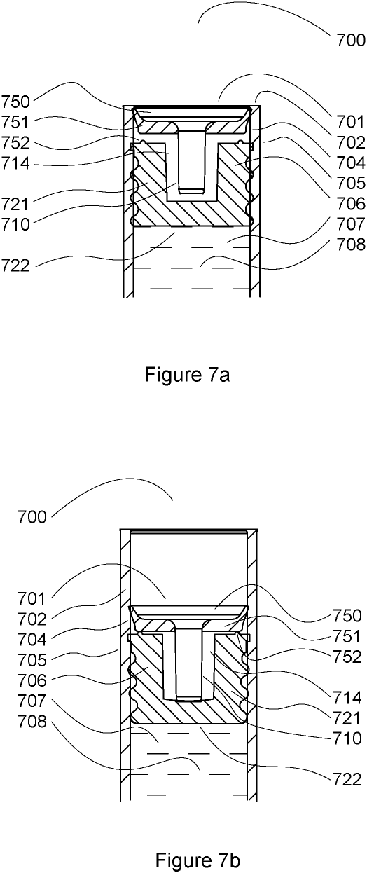

FIG. 7a illustrates an administration device 700 in which an actuator 701 may comprise a first material 750 and a second material 751, such as an elastomeric material, wherein the first material 750 may be more rigid than the second material 751. The actuator 701 may be or include a secondary stopper, including a skirt that engages a sidewall 704 to form a seal. The proximal end may comprise the first material 750 and may be open to a proximal volume of the hollow body 702. The arrangement can be configured such that the secondary stopper may be pneumatically actuated to move in the distal direction by the application of pressure to the hollow body 702. The actuator 701 may comprise a proximal end and a distal end. The distal end may comprise a post 710 extending axially. The hollow body 702 may comprise a sidewall 704 and an outer wall 705. The stopper 706 may comprise a contour 752, such as a ridge that may be configured to engage the actuator 701 to form a seal in the advancing state. Alternatively, the actuator 701 may include the contour 752 or equivalent structures, such as a gasket or matched surfaces. The stopper 706 may include an interior cavity 714 defined in the stopper. A distal volume 707 comprises a storage volume for a medicament or other agent 708, herein illustrated by shading of the region.

FIG. 7b illustrates the embodiment of FIG. 7a in which a force is applied to the stopper 706 by an actuator 701 as a result of fluid pressure, aka a pneumatic pressure, on the proximal surface of the actuator 701. Upon application of force, the post 710 may bottom out within an interior cavity 714 defined within the stopper 706 to deform and/or distally stretch a distal wall 722 of the stopper 706 and cause radial inward contraction of the stopper. The radial inward contraction may cause a reduction in a normal force acting between the stopper 706 and sidewall 704 and a reduction of a friction force acting to withstand the distal motion of the stopper 706 and actuator 701. The difference in the outward force of the stopper 706 against the sidewall 704 is shown by variation in the amount of overlap of the stopper and the sidewall. The reduced friction may allow the stopper 706 may be advanced distally with lower friction, thus promoting efficient and smooth travel of the stopper 706 relative to the sidewall 704 to dispense the medicament or other agent 708. In the implementation, the post 710 extends relative to the interior cavity 714 defined in the stopper 706. In implementations, the second stopper of the actuator 701 may be arranged distally relative to the stopper 706.

The interior cavity 714 of the stopper 706 may optionally further include a distal cavity disposed distally of a pinch point as described in other embodiments. The hollow body 702 may be cylindrical and may include a sidewall 704 and an outer wall 705, each extending axially along the hollow body 702. The post 710 may optionally include a distal knob configured to resist the inward contraction of the primary wall 721 of the stopper 706 and maintain a high static friction of the stopper 706 against the sidewall 704 in the static state. The actuator 701 may comprise a secondary stopper extending radially outward from the post 710 as described. The secondary stopper may perform in a similar manner to the flanges described in alternative embodiments. As with the flanges, the secondary stopper may have a radial cross-section greater than a radial cross-section of a proximal end of the interior cavity 714. The post 710 may extend an axial distance greater than an axial depth of the interior cavity 714, and the secondary stopper may serve to limit the axial stretching of the stopper 706 to a defined limit.

In the advancing state, the post 710 may contact the distal wall 722 of the interior cavity 714 of the stopper 706 to cause radial inward contraction of the primary wall 721 of the stopper 706, thus reducing the radial pressure of the primary wall 721 of the stopper 706 against the sidewall 704 from a first radial pressure which maintains a seal to a second pressure which may or may not maintain a seal. The breakout force may thus be reduced in the advancing state, allowing for the distal motion of the whole plunger arrangement, including the actuator 701 and stopper 706, within the hollow body 702.

In an implementation, the skirt may extend radially from the secondary stopper (as illustrated, with the skirt terminating near or substantially at a proximal end or portion of the post 710) and/or may radially extend from the post 710 (not illustrated). In an unillustrated implementation, the distal portion of the post 710 may contactingly engage the stopper 706 such that the skirt is offset from a proximal end portion of the stopper 706. The offset between the skirt and the stopper 706 may create a gap between the skirt and the stopper 706. In the advancing state, as a force acts on the actuator 701 to advance into the stopper 706 to move the stopper 706 distally relative to the sidewall 704, the second stopper may bear upon the stopper 706 to cause deformation of the stopper 706, thus reducing the gap (e.g., the amount of offset) between the skirt and the stopper 706.

While the administration device may be activated through increased pressure in a pressure chamber as described above, the arrangement of the stopper 706 and second stopper may also be under the influence of other forces, such as a mechanical force acting on the actuator 701. For instance, the actuator 701 may be engaged by or include at a distal end a rod or plunger to create the distal movement relative to the stopper 706 to achieve operation as described above. As such, the administration device 700 may be any of a jet injector, a container, a syringe, a cartridge, a barrel, or other elements with an arrangement between the actuator 701, the stopper 706, and the sidewall 704 such that the arrangement may be used in conjunction with other apparatuses such as a pump, a syringe, or other dispensing systems.

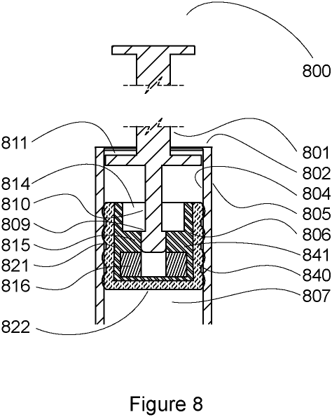

FIG. 8 illustrates an administration device 800, including an actuator 801 and a stopper 806. The actuator 801 may include a flange 811 extending radially outward. The administration device 800 may be a syringe and can include a hollow body 802 (e.g., a cylindrical barrel) with a sidewall 804 and an outer wall 805 extending axially along the hollow body 802. A distal volume 807 may serve as a storage volume for a medicament or other agent. The stopper 806 may comprise an interior cavity 814 proximal to a pinch point 815, which may contactingly engage a knob 809. The stopper 806 may include a distal cavity 816 distal to the pinch point and interior cavity 814. The stopper 806 may have an outer surface 840 and an inner surface 841, which may have different material properties or compositions. The radial wall of the distal cavity 816 of the stopper 806 may have material composition or properties which differ from one or both of the inner surface 841 and the outer surface 840.

The interior cavity 814 of the stopper 806 may optionally further include a distal cavity 816 disposed distally of the pinch point 815. The distal cavity 816 may have a radial wall with a first set of material properties. The pinch point 815 may a second set of material properties that differ from the first set. The distal cavity 816 can have a distal wall 822. The hollow body 802 may be cylindrical and may include a sidewall 804 and an outer wall 805, each extending axially along the hollow body 802. The post 810 may include a knob 809 (e.g., a distal knob on a distal portion or end of the post 810) configured to resist the inward contraction of the primary wall 821 of the stopper 806 and maintain a high static friction of the stopper 806 against the sidewall 804. The knob 809 may be the same diameter as the post 810 in some variations. The post 810 may further include a flange 811 extending radially outward and disposed proximally of the stopper. The flange 811 may have a radial cross-section greater than a radial cross-section of a proximal end of the interior cavity 814. The post 810 may longitudinally extend an axial distance greater than an axial depth of the interior cavity 814, and engagement of the radial flange with the stopper may serve to limit distal stretching of the primary wall and/or the distal wall to a predefined amount (e.g., radial distance), which may be responsive to the axial force applied to the distal wall by the post in the advancing state. The pinch point 815 may be in contact with the knob 809 in the static state.

The administration device 800 may transition between a static state, as illustrated in FIG. 8, and an advancing state, as illustrated in other embodiments of the device. The administration device 800 may pass through an intermediate configuration between the static state and the advancing state wherein the resistance to inward contraction of the primary wall 821 is removed, but the knob 809 has not yet bottomed out against the distal wall 822.

In the advancing state, the knob 809 may contact the distal wall 822 of the distal cavity 816 of the stopper 806 to cause further radial inward contraction of the primary wall 821 of the stopper 806, thus reducing the radial pressure of the primary wall 821 of the stopper 806 against the sidewall 804 from a first radial pressure which maintains a seal to a second pressure, which may or may not maintain a seal. The breakout force may thus be reduced in the advancing state, allowing for the distal motion of the whole plunger arrangement, including the actuator 801 and stopper 806, within the hollow body 802.

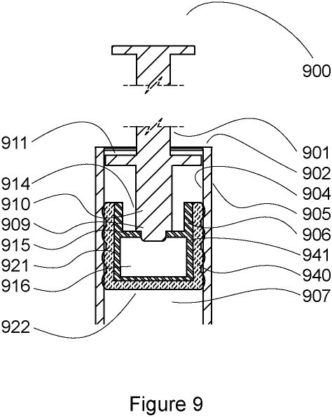

FIG. 9 illustrates an administration device 900 in which an actuator 901 comprises a post 910, which has a cross-sectional diameter greater than a knob 909 located at its distal end. The administration device 900 may include a hollow body 902, such as a cylindrical syringe barrel. The hollow body 902 may include a sidewall 904 and an outer wall 905. A distal volume 907 may serve as a storage volume for a medicament or fluid. A stopper 906 may include an interior cavity 914 proximal to a pinch point 915, which may contactingly engage the knob 909. The stopper 906 may include a distal cavity 916 distal to the pinch point and interior cavity 914. The stopper 906 may have an outer surface 940 and an inner surface 941, which may have different material properties or compositions.

The interior cavity 914 of the stopper 906 may optionally further include a distal cavity 916 disposed distally of the pinch point 915. The distal cavity 916 may have a radial wall with a first set of material properties. The pinch point 915 may have a second set of material properties that may differ from the first set. The distal cavity 916 can have a distal wall 922. The hollow body 902 may be cylindrical and may include a sidewall 904 and an outer wall 905, each extending axially along the hollow body. The post 910 may include a knob 909 (e.g., a distal knob on a distal portion or end of the post 910) configured to resist the inward contraction of the primary wall 921 of the stopper 906 and maintain a high static friction of the stopper 906 against the sidewall 904. The knob 909 may be of the same diameter as or of a lesser diameter than the post 910. The post 910 may further include a flange 911 extending radially outward and disposed proximally of the stopper. The flange 911 may have a radial cross-section greater than a radial cross-section of a proximal end of the interior cavity 914. The post 910 may longitudinally extend an axial distance greater than an axial depth of the interior cavity 914, and engagement of the radial flange with the stopper may serve to limit distal stretching of the primary wall 921 and/or the distal wall 922 to a predefined amount, which may be responsive to the axial force applied to the distal wall by the post in the advancing state. The pinch point 915 may be in contact with the knob 909 in the static state.

The administration device 900 may transition between a static state, as illustrated in FIG. 9, and an advancing state, as illustrated in other embodiments of the device. The administration device 900 may pass through an intermediate configuration between the static state and the advancing state wherein the post 910 deflects the pinch point 915 such that it may axially stretch and deform radially inward, but the knob 909 has not yet bottomed out against the distal wall 922.

In the advancing state, the knob 909 may contact the distal wall 922 of the distal cavity 916 of the stopper 906 to cause further radial inward contraction of the primary wall 921 of the stopper 906, thus reducing the radial pressure of the primary wall 921 of the stopper 906 against the sidewall 904 from a first radial pressure which maintains a seal to a second pressure which may or may not maintain a seal. The breakout force may thus be reduced in the advancing state, allowing for the distal motion of the whole plunger arrangement, including the actuator 901 and stopper 906, within the hollow body 902.

While the invention has been illustrated and described in detail in the drawings and foregoing description, such illustration and description are to be considered exemplary and not restrictive in character. For example, certain embodiments described hereinabove may be combinable with other described embodiments and/or arranged in other ways (e.g., a feature of one embodiment may be practiced or omitted in another). It should be understood that only the preferred embodiment and variants thereof have been shown and described and that all changes and modifications that come within the spirit of the invention are desired to be protected. The various features and structures of the designs should therefore be considered optional with respect to one another, and the scope of the invention is limited only by the appended claims.

What is claimed is:

1. An administration device configured to transition between a static state and an advancing state, comprising:
a hollow body including a sidewall disposed radially within the hollow body along a longitudinal length of the hollow body;
a post configured to move axially within the hollow body; and
a stopper comprising a primary wall and an interior cavity, the primary wall configured to seal against the sidewall when the post is in a first position,
wherein the stopper is configured to apply a first radial pressure against the sidewall when the post is in the first position and configured to apply a second radial pressure less than the first radial pressure against the sidewall when the post is in a second position.

2. An administration device as claimed in claim 1, wherein the interior cavity distally terminates in a distal wall, the post being configured to transition from a first position in the static state in which no axial force is applied by the post to the distal wall into a second position in the advancing state in which an axial force is applied to the distal wall by the post.

3. An administration device as claimed in claim 2, wherein the primary wall is configured to radially contract into the interior cavity in response to axial stretching of the distal wall caused by the axial force applied to the distal wall in the advancing state.

4. An administration device as claimed in claim 3, wherein the axial force is applied distally and the axial stretching is in a distal direction.

5. An administration device as claimed in claim 2, the interior cavity further comprising:
a pinch point disposed within the interior cavity, wherein the post is configured to provide a resistance to an inward contraction of the primary wall at the pinch point in the static state and the resistance is removed in the advancing state.

6. An administration device as claimed in claim 5, the post further comprising a knob with a first radial width and a rod with a second radial width greater than the first radial width, the rod configured to apply an axial force to the pinch point when the post is moved to a second position.

7. An administration device as claimed in claim 6, wherein the post is moved distally and the axial force is applied to the pinch point in a distal direction.

8. An administration device as claimed in claim 5, the interior cavity further comprising a distal cavity disposed distally of the pinch point and comprising a first radial cross-section, the pinch point comprising a second radial cross-section less than the first radial cross-section.

9. An administration device as claimed in claim 5, the post further comprising a knob with a first radial width and a rod with a second radial width equal to or less than the first radial width.

10. An administration device as claimed in claim 9, the stopper further comprising a distal cavity comprising a radial wall with material properties different from the material properties of the pinch point, the distal cavity configured to accept the first radial width when the post moves in a distal direction.

11. An administration device as claimed in claim 9, wherein the knob applies the resistance to an inward contraction in the static state at the pinch point.

12. An administration device as claimed in claim 9, wherein the knob is moved into a distal cavity in the advancing state removing the resistance at the pinch point and allowing the primary wall to contract radially inwardly around the rod, the knob configured to apply the axial force to the distal wall in the advancing state.

13. An administration device as claimed in claim 2, the post being disposed on a secondary stopper, and the secondary stopper being pneumatically actuatable.

14. An administration device as claimed in claim 2, the post further comprising:
a flange disposed proximally of the stopper, the flange having a radial cross-section greater than a radial cross-section of a proximal end of the interior cavity, the flange configured to axially engage the stopper in the advancing state.

15. An administration device as claimed in claim 14, wherein the post longitudinally extends an axial distance greater than an axial depth of the interior cavity, and engagement of the flange with the stopper limits distal stretching of the distal wall to a predefined amount from the axial force applied to the distal wall.

16. An administration device as claimed in claim 1, the post further comprising a skirt extending radially from the post.

17. An administration device as claimed in claim 16, wherein the skirt is configured to seal against the sidewall.

18. An administration device as claimed in claim 1, the post being more rigid than the stopper.

19. An administration device as claimed in claim 1, wherein the first radial pressure applied by the stopper against the sidewall provides an aseptic barrier.

20. An administration device as claimed in claim 1, wherein the post is actuated from a first position to a second position by an increase in pressure of a proximal volume.

21. An administration device as claimed in claim 1, wherein an interior surface of the primary wall includes raised surface features protruding radially inwardly into the interior cavity.

22. An administration device as claimed in claim 1, wherein a longitudinal extent of the post is an axial length greater than an axial depth of the interior cavity.
